(19) United States
(12) Patent Application Publication
(10) Pub. No.: US 2004/0258623 A1
(43) Pub. Date: Dec. 23, 2004

(54) Insulin-containing oral spray and the preparation method thereof

(76) Inventors: Huibi Xu, Wuhan (CN); Kaixun Huang, Wuhan (CN); Qiuihua Gao, Wuhan (CN); Zhonghong Gao, Wuhan (CN); Jilin Yang, Wuhan (CN)

Correspondence Address: GREENBERG TRAURIG LLP, 2450 COLORADO AVENUE, SUITE 400E, SANTA MONICA, CA, 90404, US

(21) Appl. No.: 10/486,461

(22) PCT Filed: May 20, 2002
(86) PCT No.: PCT/CN02/00342

(30) Foreign Application Priority Data
Aug. 8, 2001 (CN) 01128323.8

(51) Int. Cl. A61K 38/28; A61L 9/04; A61K 9/14

(52) U.S. Cl. 424/46; 514/3

(57) ABSTRACT

The present invention provides an insulin-containing buccal spray and the preparation method thereof. The insulin buccal spray of the invention is a microemulsion having the average diameter of less than 200 nm, comprising 10000 u-7000 u of insulin, 5-50 g of soybean lecithin as absorption promoter, 20-80 g of propylene glycol as cosolvent, and balanced with phosphate buffer of pH 6.8-7.8 to 1000 ml, based on 1000 ml microemulsion. The preparation method comprises: a) adding soybean lecithin to propylene glycol, mixing sufficiently, then adding phosphate buffer to result solution, and sonicating, thus obtaining the oil phase of the microemulsion; b) dissolving insulin in phosphate buffer, c) adding the insulin solution to the oil phase, sonicating again, thus obtaining the emulsion of the invention. The efficiency and stability of the emulsion of the invention are improved significantly, and administration of the invention is convenient.

### FIELD OF THE INVENTION

The present invention provides an insulin-containing formulation and the preparation method thereof, in particularly, an insulin-containing buccal spray for absorption through human buccal mucosa and the preparation method thereof.

### BACKGROUND OF THE INVENTION

Insulin is liable to be degraded by the gastric acid and various digestive enzymes in the gastrointestinal tract, thus can not be administrated orally. Generally, insulin is administrated by injection, and is performed at the time of half an hour before meal to regulate blood glucose. Furthermore, insulin injection would be very inconvenient since patients normally need insulin administration in their whole life. Therefore, pharmaceutical formulations having safe, convenient, and effective property, in particularly non- njection administration would be welcome by patients. The non-injection administration of insulin has become an attractive subject in pharmaceutical field worldwide in recent years. In the last ten years, great advances have been achieved. At present, there are many optional administrating routes of insulin. For example, the administrating route which insulin pump is embedded intraperitoneum is proved to be safe and effective. It has been applied clinically in some countries. However, their prices are too high to be burdened by general patients. Some investigators suggest that the method which insulin is absorbed through bronchial mucosa has attractive applying expectation. However, there are some dificulties to be solved for this method. Some studies focus on oral administration of insulin, i.e. insulin is embedded in liposomes or polymers, thus obtain oral formulation of insulin. The insulin in the formulation is absorbed through mucosal cells of small intestine to achieve the effect of decreasing blood glucose. The low bioavailability and quick clearance from the administrating site are main obstacle to oral administration of insulin. Recently, more and more researches concentrate on administrating route of nasal mucosa, ocular mucosa, pneumal mucosa, and buccal mucosa. Although some advances have been achieved upon ten years studies, the bioavailability of insulin is still low in clinical application since the molecular weight of insulin is too large to be absorbed easily. The key point is to choose suitable absorption promoter having low toxicity and high efficiency to increase bioavailability of insulin.

### DISCLOSURE OF THE INVENTION

The present invention provides an insulin-containing formulation and the preparation method thereof. The technical problem to be solved by the present invention is to further improve bioavailability of the insulin formulation absorbed through human buccal mucosa and increase stability of the formulation.

The inventor further optimizes the ratio of components of the composition and replace agitating treatment with sonicating treatment, thereby obtain a microemulsion having the average diameter of less than 200 nm based on the Chinese patent application 00114318.2.

The present invention provides an insulin buccal spray, which is microemulsion having the average diameter of less than 200 nm, comprising 10000 u-70000 u of insulin, 5-50 g of soybean lecithin as absorption promoter, 25-80 g of propylene glycol as cosolvent, and balanced with phosphate buffer of pH 6.8-7.8 to 1000 ml.

According to the insulin buccal spray of the present invention, the average diameter of the microemulsion is 100-180 nm.

The insulin buccal spray of the present invention also comprises borneol-ethanol soluton as flavoring agent and phenol as antimicrobial, wherein the amount of borneol is 1.2-10 g, the amount of absolute ethanol is 1-15 ml per 1000 ml microemulsion and the amount of phenol is 2 g-5 g per 1000 ml microemulsion.

In a preferred embodiment of the present invention, the microemulsion comprises 15000 u-60000 u insulin, 20-50 g of soybean lecithin as absorption promoter, 40-80 g of propylene glycol as cosolvent, borneol-ethanol solution as flavoring agent prepared by dissolving 1.2-10 g of borneol in 1-15 ml of absolute ethanol, 2 g-5 g of phenol as antimicrobial, and balanced with phosphate buffer of pH 6.8-7.8 to 1000 ml.

The present invention also provides a method for preparing the insulin buccal spray, comprising: adding 5-50 g of soybean lecithin to 25-80 g of propylene glycol, then adding 25-60% by volume of phosphate buffer based on the total amount of phosphate buffer, and sonicating for 0.5-2 hrs, thus obtaining an oil phase of the microemulsion; dissolving 10000 u-70000 u of insulin in 40-75% by volume of phosphate buffer based on the total amount of phosphate buffer; adding the insulin solution to the oil phase gradually, and sonicating 2-10 mins.

When the flavoring agent and antimicrobial are added, the method for preparing the insulin buccal spray according to the present invention, comprising: dissolving 2 g-5 g of phenol in phosphate buffer; adding 20-50 g of soybean lecithin to a solution of 40-80 g of propylene glycol and borneol-ethanol, then adding 25-60% by volume of phenol-containing phosphate buffer based on the total amount of phosphate buffer, and sonicating for 0.5-2 hrs, thus obtaining an oil phase of the microemulsion; dissolving 15000 u-60000 u of insulin in 40-75% by volume of phenol-containing phosphate buffer based on the total amount of phosphate buffer; adding the insulin solution to the oil phase gradually, and sonicating 2-10 mins. According to the method for preparing the insulin buccal spray of the invention, the ultrasonic frequency of sonication is 18-25 KHz, and the duty ratio of sonication is 30-90%.

According to the method for preparing the insulin buccal spray of the invention, the temperature in the processing is controlled to a range between 2° C. and 70° C.

Since insulin is a polypeptide hormone, its permeability is not good in the case of direct administration through buccal mucosa. Suitable absorption promoter must be chosen to improve bioavailability. The soybean lecithin used in the present invention is a non-toxic, safe, and effective absorption promoter. Furthermore, suitable cosolvent is required due to poor water-solubility of soybean lecithin. The effect of propylene glycol as cosolvent is investigated by the index of the hypoglycemic level in this present invention. The experiments suggest that propylene glycol can improve the effect of absorption of insulin through buccal mucosa that promoted by soybean lecithin. Their best ratio is determined by orthogonal design. The insulin in the mixture of soybean lecithin and propylene glycol form thermodynamics stable system of microemulsion. At the same time, soybean lecithin is used as surfactant and carrier of medicine. The aqueous soluble insulin is embedded in the aqueous solution of lipoidal double layers of soybean lecithin.

The insulin buccal spray of the present invention can include any pharmaceutically acceptablc excipents, as long as they do not destroy character of microemulsion and effect of medicament. These pharmaceutically acceptable excipents includes, but not be limited to various diluents, solvents, emulsifiers, preservatives, stabilizers, dissolution aids, flavoring agents, and perfuming agents.

The insulin buccal spray of the present invention has no toxicity. The long term toxicity experiment of rat was carried out by locally administrating the insulin buccal spray in which the dosages were 4.5, 9.0, 18.0 u •kg⁻¹ respectively and physiological saline and carrier were used as control. No obvious abnormality was observed.

The insulin buccal spray prepared by sonicatng has significantly improved efficiency and stability as compared with those prepared by previous methods, such as the method described in the Chinese patent application No.00114318.2.

1. Stability. The insulin buccal spray prepared by sonicating according to the invention has significantly smaller diameter of the microemulsion, i.e. nanometer grade, in particularly the average diameter is less than 200 nm and distribution of size is between 100 nm and 160 nm, while the average diameter of previous microemulsion are about 427.2 nm. The nanometer grade microemulsion not only favor to absorption through mucosa, but also improve stability of the formulation. The insulin buccal spray prepared by sonicatng is translucent at the time of visual inspection. After placed in a freezer of 2-8° C. for 1 year, the insulin buccal spray of the invention does not form precipitate. On the contrary, the insulin buccal spray prepared by agitation previously is opaque, and form slight precipitate after placed in a freezer of 2-8° C. for 1 year. Therefore, the sonicating treatment is very important to the preparation of microemulsion.

2. Efficiency. The insulin content in vivo in which normal rabbits are used as pharmacological model are determined by enzyme-linked immunoassay. The pharmacokinetic parameter of the insulin buccal spray according to the present invention is calculated. The result suggests that the serum insulin concentration rise rapidly after administrating to normal rabbits through buccal mucosa with the dosage of 1.5 u •kg⁻¹ body weight. The peak time is in the range of between 55 mins and 70 mins and the peak concentration up to 145.2±5.8 μ/ml. The serum concentration is decreased to base level after 46 hrs. The bioavailability is up to 29.8%. Compared with the control groups without insulin, the groups of administrating insulin through buccal mucosa and those of administrating insulin by subcutaneous injection both show significant difference in the serum insulin concentration of rabbit within 30-120 mins (p<0.05). The groups of administrating insulin through buccal mucosa (1.5 u •kg⁻¹)and the control groups of administrating insulin by subcutaneous in ection (0.5 u •kg⁻¹) have substantively the same peak concentration and peak time, and have no significant difference (p>0.1).

The insulin buccal spray of the present invention can be administrated in single dosage or be divided several dosages, preferably, administrated three times per day (before breakfast, lunch, and supper) or four times per day (before breakfast, lunch, supper, and sleeping). The insulin buccal spray is administrated at the time of one hour before meal. The dosages of the insulin buccal spray of the present invention have no specific limitation and can be administrated based on conventional dosages of insulin. The particular dosages will vary in accordance with individual patients, bioavailability, and severity of conditions, and should be determined by the physicians.

The insulin buccal spray of the present invention is a microemulsion, which has the advantages of large contact area with buccal mucosa, rapid absorption, safety, and excellent hypoglycemic action. The formulation is packaged in a bottle with constant delivery pump, which spray the haze of the formulation to buccal cavity.

The insulin is adhered to mucosa of buccal cavity and absorbed rapidly through mucosa to circulation. Thus the rate of absorption is improved significantly. Furthermore, the acidolysis and enzymolysis through gastrointestinal tract and first pass effect of liver can be avoided. The insulin buccal spray of the present invention is a new non-injectable administration mode, which is convenient to patients. The formulation relieves inconvenience and pain of patients suffered diabetes and thus has perfect practicability.

### EXAMPLES

### Example 1

### 1000 ml Insulin Microemulsion

| | | |
|---|---|---|
| insulin | 40000 | u |
| soybean lecithin | 25.0 | g |
| propylene glycol | 75.0 | g |
| phosphate buffer (pH = 7.4) | q.s. to 1000 | ml |

According to above formulating ratio, 25.0 g of soybean lecithin was added to 75.0 g of propylene glycol, mixed sufficiently to thin paste. 550 ml of phosphate buffer (pH=7.4) was added. Above components were used as carrier of the formulation. The carrier then was sonicated for 1 hr (Condition: the duty ratio of sonication was 50%, the ultrasonic frequency of sonication was 20 KHz, and the temperature was controlled to a range between 40° C. and 60° C. 4000 u of insulin was dissolved in 350 ml of phosphate buffer (pH=7.4), then slowly added to sonicated carrier solution as prepared above, and continued to sonicate for 5 mins. The average diameter was 160-180 nm determined by laser granularity test instrument.

The insulin buccal spray of the present invention was prepared in the strictly aseptic condition. The spray prepared was placed in a freezer of 2-8° C. for storage.

The insulin buccal spray prepared above has excellent hypoglycemic action. When the diabetic rats induced by streptozotocin were administrated through buccal mucosa with the dosage of 1, 3, 9 u •kg⁻¹, the hypoglycemic ratios were 20.9%, 47.6%, 58.8% respectively. When the diabetic rabbits induced by alloxan were administrated through buccal mucosa with the dosage of 5.5, 1.5, 4.5 u •kg⁻¹, the hypoglycemic ratios were 24.9%, 52.6%, 60.9% respectively.

### Example 2

### 1000 ml Insulin Microemulsion

| | | |
|---|---|---|
| insulin | 20000 | u |
| soybean lecithin | 30.0 | g |
| propylene glycol | 45.0 | g |
| borneol | 1.2 | g |
| absolute ethanol | 3.0 | ml |
| phenol | 2.0 | g |
| phosphate buffer (pH = 7.4) | q.s. to 1000 | ml |

According to above formulating ratio, phenol was dissolved in phosphate buffer. 30.0 g of soybean lecithin was added to 45.0 g, of propylene glycol and borneol-ethanol solution. Mixed sufficiently, then 555 ml of phenol-containing phosphate buffer (pH=7.4) was added. Above components were used as carrier of the formulation. The carrier then was sonicated for 1.5 hrs (Condition: the duty ratio of sonicating was 60%, ultrasonic frequency of sonicating was 18 KHz, and the temperature was controlled to a range between 50° C. and 60° C.). 20000 u of insulin was dissolved in 345 ml of phenol-containing phosphate buffer (pH=7.4), then slowly added to sonicated carrier solution as prepared above, and continued to sonicate for 4 mins. The average diameter was 160-180 nm determined by laser granularity test instrument.

The insulin buccal spray as prepared above was placed in a freezer of 2-8° C. for 1 year. No precipitate was formed.

The insulin buccal spray prepared above has excellent hypoglycemic action. When the diabetic rats induced by streptozotocin were administrated through buccal mucosa with dosage of 1, 3, 9 u •kg⁻¹, the hypoglycemic ratios were 21.8%, 47.2%, 56.2% respectively. When the diabetic rabbits induced by alloxan were administrated through buccal mucosa with dosage of 0.5, 1.5. 4.5 u •kg⁻¹, the hypoglycemic ratios were 28.6%, 55.2%, 60.7% respectively.

### Example 3

### 1000 ml Insulin Microemulsion

| | | |
|---|---|---|
| insulin | 400000 | u |
| soybean lecithin | 25.0 | g |
| propylene glycol | 75.0 | g |
| bomeol | 1.2 | g |
| absolute ethanol | 4.0 | ml |
| phenol | 2.0 | g |
| phosphate buffer (pH = 7.0) | q.s. to 1000 | ml |

According to above formulating ratio, 25.0 g of soybean lecithin was added to 75.0 g of propylene glycol and borneol-ethanol solution, mixed sufficiently. 540 ml of phenol containing phosphate buffer (pH=7.0) then was added. Above components were used as carrier of the formulation. The carrier was sonicated for 1 hr (Condition: the duty ratio of sonicating was 50%, ultrasonic frequency of sonicating was 20 KHz, and the temperature was controlled to a range between 40° C. and 55° C.). 40000 u of insulin was dissolved in 360 ml of phenol-containing phosphate buffer (pH=7.0), then slowly added to sonicated carrier solution as prepared above, and continued to sonicate for 5 mins. The average diameter was 130-150 nm determined by laser granularity test instrument.

The insulin buccal spray as prepared above was placed in a freezer of 2-8° C. for 1 year. No precipitate was formed.

The insulin buccal spray prepared above has excellent hypoglycemic action. When the diabetic rats induced by streptozotocin were administrated through buccal mucosa with dosage of 1, 3, 9 u •kg⁻¹, the hypoglycemic ratios were 21.3%, 49.8%, 60.2% respectively. When the diabetic rabbits induced by alloxan were administrated through buccal mucosa with dosage of 0.5, 1.5, 4.5 u •kg⁻¹, the hypoglycemic ratios were 29.6%, 55.4%, 62.2% respectively.

### Example 4

### 1000 ml Insulin Microemulsion

| | | |
|---|---|---|
| insulin | 60000 | u |
| soybean lecithin | 35.5 | g |
| propylene glycol | 80.0 | g |
| borneol | 1.4 | g |
| absolute ethanol | 4.0 | ml |
| phenol | 2.0 | g |
| phosphate buffer (pH = 7.8) | q.s. to 1000 | ml |

According to above formulating ratio, 35.5 g of soybean lecithin was added to 80.0 g of propylene glycol and borneol-ethanol solution, and mixed sufficiently. 545 ml of phenol-containing phosphate buffer (pH=7.8) then was added. Above components were used as carrier of the formulation. The carrier was sonicated for 2 hrs (Condition: the duty ratio of sonicating was 70%, ultrasonic frequency of sonicating was 25 KHz, and the temperature was controlled to a range between 55 ° C. and 70° C.). 60000 u of insulin was dissolved in 360 ml of phenol-containing phosphate buffer (pH=7.8), then slowly added to sonicated carrier solution as prepared above, and continued to sonicate for 3 mins. The average diameter was 140-160 nm determined by laser granularity test instrument.

The insulin buccal spray as prepared above was placed in a freezer of 2-8° C. for 1 year. No precipitate was formed.

The insulin buccal spray prepared above has excellent hypoglycemic action. When the diabetic rats induced by steptozotocin were administrated through buccal mucosa with dosage of 1, 3, 9 u •kg⁻¹, the hypoglycemic ratios were 18.3%, 35.2%, 44.2% respectively. When the diabetic rabbits induced by alloxan were administrated through buccal mucosa with dosage of 0.5, 1.5, 4.5 u •kg⁻¹, the hypoglycemic ratios were 20.1%, 45.4%, 52.2% respectively.

What is claimed is:

1. An insulin bucca spray, which is microemulsion having the average diameter of less than 200 nm, comprising 10000 u-70000 u of insulin, 5-50 g of soybean lecithin as absorption promoter, 25-80 g of propylene glycol as cosolvent, and balanced with phosphate buffer of pH 6.8-7.8 to 1000 ml.

2. The insulin buccal spray according to claim 1, which comprises 15000 u-60000 u of insulin, 20-50 g of soybean lecithin as absorption promoter, 40-80 g of propylene glycol as cosolvent, and balanced with phosphate buffer of pH 6.8-7.8 to 1000 ml.

3. The insulin buccal spray according to claim 1, wherein the average diameter of the microemulsion is 100-180 nm.

4. The insulin buccal spray according to claim 2, wherein the microemulsion also comprises borneol-ethanol solution as flavoring agent, and the amount of borneol is 1.2-10 g, the amount of absolute ethanol is 1-15 ml per 1000 ml microemulsion.

5. The insulin buccal spray according to claim 2, wherein the microemulsion also comprises phenol as antimicrobial, and the amount of phenol is 2 g-5 g per 1000 ml microemulsion.

6. A method for preparing the insulin buccal spray according to any one of claims 1-5, comprising:
adding 5-50 g of soybean lecithin to 25-80 g of propylene glycol, then adding 25-60% by volume of phosphate buffer based on the total amount of phosphate buffer, and sonicating for 0.5-2 hrs, thus obtaining an oil phase of the microemulsion;
dissolving 10000 u-70000 u of insulin in 40-75% by volume of phosphate buffer based on the total amount of phosphate buffer;
adding the insulin solution to the oil phase gradually, and sonicating 2-10 mins.

7. The method according to claim 6, comprising:
dissolving 2 g-5 g of phenol in phosphate buffer;
adding 20-50 g of soybean lecithin to a solution of 40-80 g of propylene glycol and borneol-ethanol, then adding 25-60% by volume of phenol-containing phosphate buffer based on the total amount of phosphate buffer, and sonicating for 0.52 hrs, thus obtaining ar oil phase of the microemulsion;
dissolving 15000 u-30000 u of insulin in 40-75% by volume of phenol-containing phosphate buffer based on the total amount of phosphate buffer;
adding the insulin solution to the oil phase gradually, and sonicating 2-10 mins.

8. The method according to claim 6, wherein the ultrasonic frequency of sonication is 18-25 KHz, and the duty ratio of sonication is 30-90%.

9. The method according to claim 7, wherein the ultrasonic frequency of sonication is 18-25 KHz, and the duty ratio of sonitation is 30-90%.

10. The method according to claim 6, wherein the temperature in the processing is controlled to a range between 2° C. and 70° C.

11. The method according to claim 7, wherein the temperature in the processing is controlled to a range between 2° C. and 70° C.
